(19) United States
(12) Patent Application Publication
(10) Pub. No.: US 2019/0139628 A1
(43) Pub. Date: May 9, 2019

(54) MACHINE LEARNING TECHNIQUES FOR ANALYSIS OF STRUCTURAL VARIANTS

(71) Applicant: Arc Bio, LLC, Cambridge, MA (US)

(72) Inventors: Thomas J. Watson, Jr., Auburndale, MA (US); Alejandro Quiroz Zarate, Cambridge, MA (US)

(73) Assignee: Arc Bio, LLC, Cambridge, MA (US)

(21) Appl. No.: 16/096,114

(22) PCT Filed: Apr. 26, 2017
(86) PCT No.: PCT/US17/29563

§ 371 Date: Oct. 24, 2018

Related U.S. Application Data
(60) Provisional application No. 62/328,240, filed on Apr. 27, 2016.

(51) Int. Cl. G16B 40/00; G16B 30/10; G06N 20/20

(52) U.S. Cl. CPC G16B 40/00; G06N 20/20; G16B 30/10

(57) ABSTRACT

The present disclosure provides techniques for analysis of genetic features. In particular, machine learning techniques can be used to analyze various statistical features in determining genetic features such as variants, markers, and traits, for example in a nucleotide sequence.

### CROSS-REFERENCE

This application claims the benefit of U.S. Provisional Application No. 62/328,240, filed Apr. 27, 2016, which is hereby incorporated by reference in its entirety for all purposes.

### BACKGROUND

Even though DNA is formed through the interaction of four nucleotides, the combined 3 billion-long string of nucleotides that constitutes the human genome contains important analytical challenges. One of these challenges is the inference and prediction of variants including structural variants.

Structural variants come in many forms, sizes, combinations throughout the entire genome. These include, but are not limited to, deletions, insertions, and inversions.

### SUMMARY

Techniques described in the present disclosure can be used for a variety of applications, including but not limited to integration with genome analysis tools. Alignment data, including linear and graph alignments, can be analyzed with the techniques described herein. Analysis can be performed to assess, detect, predict, characterize, or otherwise analyze genetic features, including but not limited to variants, markers, traits, and other features. Variants can include structural variants, such as deletions, insertions, and inversions. Structural variants can be classified according to their length: short: (6 bp≤SV<50 bp), medium: (50 bp≤SV<500 bp), and large: (500 by≤SV).

Analysis can be conducted using machine learning trained algorithms. For example, trained algorithms can be used to predict the presence of structural variants based on analysis of one or more statistical features. In some cases, an aligner can consider at the most 5 errors on the alignment.

In a first aspect, provided herein is a method for detecting a genetic feature in a nucleotide sequence. The method can comprise or consist essentially of: (a) analyzing aligned reads from the nucleotide sequence for at least one statistical feature selected from the group consisting of:

percent AT content,

percent GC content,

percent of soft clips,

percent of hard clips,

percent of reads with insert size greater than q₀ quartile,

percent of reads with insert size less than q₀ quartile,

percent of positive strand reads,

percent of negative strand reads,

percent of reads with a correct orientation,

percent of reads with a 0 x 1 BAM flag,

percent of reads with a 0 x 2 BAM flag,

percent of reads with a 0 x 4 BAM flag,

percent of reads with a 0 x 8 BAM flag,

percent of reads with a 0 x 20 BAM flag,

percent of reads with a 0 x 40 BAM flag,

percent of reads with a 0 x 80 BAM flag,

percent of reads with a 0 x 100 BAM flag,

percent of reads with a 0 x 200 BAM flag,

percent of reads with a 0 x 400 BAM flag, and

percent of reads with a 0 x 800 BAM flag; and

(b) based on the analyzing, determining a presence of the genetic feature in the nucleotide sequence. The aligned reads can be contained in an unsorted file. The analyzing can be performed using a programmed computer. The analyzing can be performed using a trained algorithm. The trained algorithm can comprise a random forest algorithm. The trained algorithm can be trained using a moving window. The analyzing can be performed using a moving window. The moving window can have a length of about 50 bp. The moving window can have a variable length. In some cases, the analyzing does not include portions of the aligned reads located outside the moving window. The at least one statistical feature can comprise at least two statistical features. The at least one statistical feature can comprise at least five statistical features. The presence of the genetic feature can be determined within a window of about 50 base pairs (bp). The genetic feature can be a structural variant. The structural variant can be selected from the group consisting of deletions, insertions, and inversions. The genetic feature can be a pathogenicity marker. The genetic feature can be a resistance marker. The genetic feature can be a susceptibility marker. The genetic feature can be a taxonomic marker. The genetic feature can be from about 6 base pairs (bp) to about 50 bp in length. The genetic feature can be from about 50 base pairs (bp) to about 500 bp in length. The genetic feature can be greater than about 500 base pairs in length. The presence of the genetic feature can be determined with at least 95% confidence. The presence of the genetic feature can be determined with at least 95% accuracy. The presence of the genetic feature can be determined with at least 95% specificity. The presence of the genetic feature can be determined with at least 95% sensitivity. The determining the presence of the genetic feature can comprise determining the presence of a start or an end of the genetic feature. The aligned reads can comprise graph aligned reads. The analyzing can be performed using a moving window, further comprising analyzing the graph aligned reads for at least one statistical feature selected from the group consisting of: percent of paths or bubbles that fall within the window, percent of start of the path or bubbles that fall within the window, percent of ends of the path or bubbles that fall within the window, percent of complete sections of the path or bubbles that fall within the window, mean depth for each path or bubble that fall within the window, a statistical significance of each path or bubble that falls within the window, a portion of a total length of each path or bubble that falls within the window, and VCF file information for each path or bubble that falls within the window.

The aligned reads can align to regions with no alternative paths. The aligned reads can align to regions with no bubbles. The aligned reads can align to regions with at least one alternative path or bubble.

In another aspect, provided herein is a method for detecting a genetic feature in a nucleotide sequence, comprising: (a) analyzing aligned reads from the nucleotide sequence for at least one statistical feature; and (b) based on the analyzing, determining a presence of the genetic feature in the nucleotide sequence, wherein the genetic feature is a structural variant selected from the group consisting of an insertion, a deletion, and an inversion. The aligned reads can be contained in an unsorted file. The genetic feature can be from about 6 base pairs (bp) to about 50 bp in length. The genetic feature can be from about 50 base pairs (bp) to about 500 bp in length. The genetic feature can be greater than about 500 base pairs in length. The analyzing can be performed using a programmed computer. The analyzing can be performed using a trained algorithm. The trained algorithm can comprise a random forest algorithm. The trained algorithm can be trained using a moving window. The analyzing can be performed using a moving window. The moving window can have a length of about 50 bp. The moving window can have a variable length. In some cases, the analyzing does not include portions of the aligned reads located outside the moving window. The at least one statistical feature can comprise at least two statistical features. The at least one statistical feature can comprise at least five statistical features. The presence of the genetic feature can be determined within a window of about 50 base pairs (bp). The at least one statistical feature can be selected from the group consisting of percent AT content, percent GC content, percent of soft clips, percent of hard clips, percent of reads with insert size greater than q₀ quartile, percent of reads with insert size less than q₀ quartile, percent of positive strand reads, percent of negative strand reads, percent of reads with a correct orientation, percent of reads with a 0 x 1 BAM flag, percent of reads with a 0 x 2 BAM flag, percent of reads with a 0 x 4 BAM flag, percent of reads with a 0×8 BAM flag, percent of reads with a 0 x 20 BAM flag, percent of reads with a 0 x 40 BAM flag, percent of reads with a 0 x 80 BAM flag, percent of reads with a 0 x 100 BAM flag, percent of reads with a 0 x 200 BAM flag, percent of reads with a 0 x 400 BAM flag, and percent of reads with a 0 x 800 BAM flag.

The at least one statistical feature can be selected from the group consisting of: number of paths or bubbles that fall within a window of width w, number of beginnings of paths or bubbles that fall within a window of width w, number of ends of paths or bubbles that fall within a window of width w, number of complete sections of paths or bubbles that fall within a window of width w, mean depth of paths or bubbles that fall within a window of width w, significance of paths or bubbles that fall within a window of width w, portion of a total length of each path of bubble that falls within a window of width w, and VCF file information for each path or bubble that falls within a window of width w. The presence of the genetic feature can be determined with at least 95% confidence. The presence of the genetic feature can be determined with at least 95% accuracy. The presence of the genetic feature can be determined with at least 95% specificity. The presence of the genetic feature can be determined with at least 95% sensitivity. The determining the presence of the genetic feature can comprise determining the presence of a start or an end of the genetic feature. The aligned reads can comprise graph aligned reads. The analyzing can be performed using a moving window, further comprising analyzing the graph aligned reads for at least one statistical feature selected from the group consisting of: percent of paths or bubbles that fall within the window, percent of start of the path or bubbles that fall within the window, percent of ends of the path or bubbles that fall within the window, percent of complete sections of the path or bubbles that fall within the window, mean depth for each path or bubble that fall within the window, a statistical significance of each path or bubble that falls within the window, a portion of a total length of each path or bubble that falls within the window, and VCF file information for each path or bubble that falls within the window. The aligned reads can align to regions with no alternative paths. The aligned reads can align to regions with no bubbles. The aligned reads can align to regions with at least one alternative path or bubble.

In another aspect, provided herein is method for detecting a genetic feature in a nucleotide sequence, comprising: (a) analyzing aligned reads from the nucleotide sequence for at least one statistical feature, wherein the analyzing is performed using a trained algorithm that employs a moving window, and wherein the analyzing does not include portions of the aligned reads located outside the moving window; and (b) based on the analyzing, determining a presence of the genetic feature in the nucleotide sequence. The aligned reads can be contained in an unsorted file. The genetic feature can be from about 6 base pairs (bp) to about 50 bp in length. The genetic feature can be from about 50 base pairs (bp) to about 500 bp in length. The genetic feature can be greater than about 500 base pairs in length. The analyzing can be performed using a programmed computer. The trained algorithm can comprise a random forest algorithm. The genetic feature can be a structural variant. The structural variant can be selected from the group consisting of deletions, insertions, and inversions. The genetic feature can be a pathogenicity marker. The genetic feature can be a resistance marker. The genetic feature can be a susceptibility marker. The genetic feature can be a taxonomic marker. The moving window can have a length of about 50 bp. The moving window can have a variable length. The at least one statistical feature can comprise at least two statistical features. The at least one statistical feature can comprise at least five statistical features. The presence of the genetic feature can be determined within a window of about 50 base pairs (bp). The at least one statistical feature can be selected from the group consisting of: percent AT content, percent GC content, percent of soft clips, percent of hard clips, percent of reads with insert size greater than q₀ quartile, percent of reads with insert size less than q₀ quartile, percent of positive strand reads, percent of negative strand reads, percent of reads with a correct orientation, percent of reads with a 0 x 1 BAM flag, percent of reads with a 0 x 2 BAM flag, percent of reads with a 0 x 4 BAM flag, percent of reads with a 0 x 8 BAM flag, percent of reads with a 0×20 BAM flag, percent of reads with a 0 x 40 BAM flag, percent of reads with a 0 x 80 BAM flag, percent of reads with a 0 x 100 BAM flag, percent of reads with a 0 x 200 BAM flag, percent of reads with a 0 x 400 BAM flag, and percent of reads with a 0×800 BAM flag. The at least one statistical feature can be selected from the group consisting of: number of paths or bubbles that fall within a window of width w, number of beginnings of paths or bubbles that fall within a window of width w, number of ends of paths or bubbles that fall within a window of width w, number of complete sections of paths or bubbles that fall within a window of width w, mean depth of paths or bubbles that fall within a window of width w, significance of paths or bubbles that fall within a window of width w, portion of a total length of each path of bubble that falls within a window of width w, and VCF file information for each path or bubble that falls within a window of width w. The presence of the genetic feature can be determined with at least 95% confidence. The presence of the genetic feature can be determined with at least 95% accuracy. The presence of the genetic feature can be determined with at least 95% specificity. The presence of the genetic feature can be determined with at least 95% sensitivity. The determining the presence of the genetic feature can comprise determining the presence of a start or an end of the genetic feature. The aligned reads can comprise graph aligned reads. The analyzing can be performed using a moving window, further comprising analyzing the graph aligned reads for at least one statistical feature selected from the group consisting of: percent of paths or bubbles that fall within the window, percent of start of the path or bubbles that fall within the window, percent of ends of the path or bubbles that fall within the window, percent of complete sections of the path or bubbles that fall within the window, mean depth for each path or bubble that fall within the window, a statistical significance of each path or bubble that falls within the window, a portion of a total length of each path or bubble that falls within the window, and VCF file information for each path or bubble that falls within the window. The aligned reads can align to regions with no alternative paths. The aligned reads can align to regions with no bubbles. The aligned reads can align to regions with at least one alternative path or bubble.

In another aspect, provided herein is a method for detecting a genetic feature in a nucleotide sequence. The method can comprise or consist essentially of: (a) analyzing graph aligned reads from the nucleotide sequence for at least one statistical feature; and (b) based on the analyzing, determining a presence of the genetic feature in the nucleotide sequence. The aligned reads can be contained in an unsorted file. The genetic feature can be from about 6 base pairs (bp) to about 50 bp in length. The genetic feature can be from about 50 base pairs (bp) to about 500 bp in length. The genetic feature can be greater than about 500 base pairs in length. The analyzing can be performed using a programmed computer. The analyzing can be performed using a trained algorithm. The trained algorithm can comprise a random forest algorithm. The trained algorithm can be trained using a moving window. The analyzing can be performed using a moving window. The moving window can have a length of about 50 bp. The moving window can have a variable length. In some cases, the analyzing does not include portions of the aligned reads located outside the moving window. The at least one statistical feature can comprise at least two statistical features. The at least one statistical feature can comprise at least five statistical features. The presence of the genetic feature can be determined within a window of about 50 base pairs (bp). The at least one statistical feature can be selected from the group consisting of: percent AT content, percent GC content, percent of soft clips, percent of hard clips, percent of reads with insert size greater than q₀ quartile, percent of reads with insert size less than q₀ quartile, percent of positive strand reads, percent of negative strand reads, percent of reads with a correct orientation, percent of reads with a 0 x 1 BAM flag, percent of reads with a 0 x 2 BAM flag, percent of reads with a 0 x 4 BAM flag, percent of reads with a 0×8 BAM flag, percent of reads with a 0 x 20 BAM flag, percent of reads with a 0 x 40 BAM flag, percent of reads with a 0 x 80 BAM flag, percent of reads with a 0 x 100 BAM flag, percent of reads with a 0 x 200 BAM flag, percent of reads with a 0 x 400 BAM flag, and percent of reads with a 0 x 800 BAM flag. The at least one statistical feature can be selected from the group consisting of: number of paths or bubbles that fall within a window of width w, number of beginnings of paths or bubbles that fall within a window of width w, number of ends of paths or bubbles that fall within a window of width w, number of complete sections of paths or bubbles that fall within a window of width w, mean depth of paths or bubbles that fall within a window of width w, significance of paths or bubbles that fall within a window of width w, portion of a total length of each path of bubble that falls within a window of width w, and VCF file information for each path or bubble that falls within a window of width w. The presence of the genetic feature can be determined with at least 95% confidence. The presence of the genetic feature can be determined with at least 95% accuracy. The presence of the genetic feature can be determined with at least 95% specificity. The presence of the genetic feature can be determined with at least 95% sensitivity. The determining the presence of the genetic feature can comprise determining the presence of a start or an end of the genetic feature. The genetic feature can be a structural variant. The structural variant can be selected from the group consisting of an insertion, a deletion, and an inversion. The genetic feature can be a pathogenicity marker. The genetic feature can be a resistance marker. The genetic feature can be a susceptibility marker. The genetic feature can be a taxonomic marker. The aligned reads can align to regions with no alternative paths. The graph aligned reads can align to regions with no bubbles. The graph aligned reads can align to regions with at least one alternative path or bubble.

In a further aspect, provided herein is a method for detecting a genetic feature in a nucleotide sequence. The method can comprise or consist essentially of (a) analyzing, using a moving window, aligned reads from the nucleotide sequence for at least one statistical feature selected from the group consisting of: number of paths or bubbles that fall within the window, number of beginnings of paths or bubbles that fall within the window, number of ends of paths or bubbles that fall within the window, number of complete sections of paths or bubbles that fall within the window, mean depth of paths or bubbles that fall within the window, significance of paths or bubbles that fall within the window, portion of a total length of each path of bubble that falls within the window, and VCF file information for each path or bubble that falls within the window; and (b) based on the analyzing, determining a presence of the genetic feature in the nucleotide sequence. The aligned reads can be contained in an unsorted file. The analyzing can be performed using a programmed computer. The analyzing can be performed using a trained algorithm. The trained algorithm can comprise a random forest algorithm. The trained algorithm can be trained using a moving window. The analyzing can be performed using a moving window. The moving window can have a length of about 50 bp. The moving window can have a variable length. In some cases, the analyzing does not include portions of the aligned reads located outside the moving window. The at least one statistical feature can comprise at least two statistical features. The at least one statistical feature can comprise at least five statistical features. The presence of the genetic feature can be determined within a window of about 50 base pairs (bp). The genetic feature can be a structural variant. The structural variant can be selected from the group consisting of deletions, insertions, and inversions. The genetic feature can be a pathogenicity marker. The genetic feature can be a resistance marker. The genetic feature can be a susceptibility marker. The genetic feature can be a taxonomic marker. The genetic feature can be from about 6 base pairs (bp) to about 50 bp in length. The genetic feature can be from about 50 base pairs (bp) to about 500 bp in length. The genetic feature can be greater than about 500 base pairs in length. The presence of the genetic feature can be determined with at least 95% confidence. The presence of the genetic feature can be determined with at least 95% accuracy. The presence of the genetic feature can be determined with at least 95% specificity. The presence of the genetic feature can be determined with at least 95% sensitivity. The determining the presence of the genetic feature can comprise determining the presence of a start or an end of the genetic feature. The aligned reads can comprise graph aligned reads. The analyzing can be performed using a moving window, further comprising analyzing the graph aligned reads for at least one statistical feature selected from the group consisting of: percent of paths or bubbles that fall within the window, percent of start of the path or bubbles that fall within the window, percent of ends of the path or bubbles that fall within the window, percent of complete sections of the path or bubbles that fall within the window, mean depth for each path or bubble that fall within the window, a statistical significance of each path or bubble that falls within the window, a portion of a total length of each path or bubble that falls within the window, and VCF file information for each path or bubble that falls within the window. The aligned reads can align to regions with no alternative paths. The aligned reads can align to regions with no bubbles. The aligned reads can align to regions with at least one alternative path or bubble. The method can further comprise analyzing aligned reads from the nucleotide sequence for at least one statistical feature selected from the group consisting of: percent AT content, percent GC content, percent of soft clips, percent of hard clips, percent of reads with insert size greater than q₀ quartile, percent of reads with insert size less than q₀ quartile, percent of positive strand reads, percent of negative strand reads, percent of reads with a correct orientation, percent of reads with a 0 x 1 BAM flag, percent of reads with a 0 x 2 BAM flag, percent of reads with a 0 x 4 BAM flag, percent of reads with a 0×8 BAM flag, percent of reads with a 0 x 20 BAM flag, percent of reads with a 0 x 40 BAM flag, percent of reads with a 0 x 80 BAM flag, percent of reads with a 0 x 100 BAM flag, percent of reads with a 0 x 200 BAM flag, percent of reads with a 0 x 400 BAM flag, and percent of reads with a 0 x 800 BAM flag.

In another aspect, provided herein is a method for detecting a genetic feature in a nucleotide sequence. The method can comprise or consist essentially of analyzing aligned reads from the nucleotide sequence for at least one statistical feature selected from the group consisting of input information depth, coverage, orientation of the aligned reads, and insert size between paired-end reads; and based on the analyzing, determining a presence of the genetic feature. The aligned reads can be contained in an unsorted file. The genetic feature can be a clade marker. The clade marker can be a pathogen clade marker. The clade marker can be a bacteria clade marker. The clade marker can be a virus clade marker. The clade marker can be a fungus clade marker. The clade marker can be a protozoa clade marker. The genetic feature can be a structural variant. The structural variant can be an insertion. The structural variant can be a deletion. The structural variant can be a copy number variation. The structural variant can be an inversion. The method can further comprise, based on the analyzing, determining a location of the genetic feature. The method can further comprise determining a confidence value of the location of the genetic feature. The genetic feature can be a structural variant. The genetic feature can be a flanking region. The analyzing can be performed using a moving window. The moving window can have a variable length. In some cases, the analyzing does not include portions of the aligned reads located outside of the moving window. The aligned reads can comprise graph aligned reads. The aligned reads can align to regions with no alternative paths. The aligned reads can align to regions with no bubbles. The aligned reads can align to regions with at least one alternative path or bubble.

In a further aspect, provided herein is a method for locating a genetic feature in a nucleotide sequence. The method can comprise or consist essentially of analyzing prior information, the prior information comprising (i) genetic feature population information or (ii) genetic feature reference information; analyzing genetic feature presence information; based on the analyzing in (a) and the analyzing in (b), determining a location of the genetic feature. The aligned reads can be contained in an unsorted file. The genetic feature presence information can be determined by analyzing aligned reads from the nucleotide sequence for at least one statistical feature and, based on the analyzing, determining the presence of the genetic feature. The method can further comprise determining a confidence value of the location of the genetic feature. The genetic feature can be a clade marker. The clade marker can be a pathogen clade marker. The clade marker can be a bacteria clade marker. The clade marker can be a virus clade marker. The clade marker can be a fungus clade marker. The clade marker can be a protozoa clade marker. The genetic feature can be a structural variant. The structural variant can be an insertion. The structural variant can be a deletion. The structural variant can be a copy number variation. The structural variant can be an inversion. The method can further comprise determining a location of each of a plurality of genetic features. The method can further comprise a confidence value for each location of the plurality of genetic features. The method can further comprise determining a genomic structure of the plurality of genetic features.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF FIGURES

FIG. 1 shows an exemplary schematic of reads aligned to a reference genome or graph.

FIG. 2A and FIG. 2B show an exemplary schematic of reads considered within the two different windows, from which extract the relevant statistics can be extracted.

FIG. 3A and FIG. 3B show an exemplary schematic of reads with bubbles.

FIG. 4 shows an exemplary schematic of modules programmed or otherwise configured to implement the methods provided herein.

FIG. 5 shows an exemplary computer system that is programmed or otherwise configured to implement the methods provided herein.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The below terms are discussed below to illustrate meanings of the terms as used in this specification, in addition to the understanding of these terms by of those of skill in the art. As used in the specification and claims, the singular forms “a”, “an” and “the” can include plural references unless the context clearly dictates otherwise. For example, the term “a cell” can include a plurality of cells, including mixtures thereof

As used herein, the term “alignment” can be any computational process in which every sequence strings produced by a sequencer is matched to a reference string. An alignment can be, for example, a Smith Waterman local alignment, a gapped alignment or semi-gapped alignment.

Variability in the genome can be represented as “alternative paths.” For example a primary genome can be a linear sequence of DNA bases (represented by the letters A, C, T, and G). A secondary genome may have a different sequence of DNA bases which represents the biological diversity between the primary and secondary subject.

“Correlated loci” can mean sequences from two genomes, or a subject genome and a reference genome, which generally represent the same genomic region. It can also mean sequences from one genome but two or more different regions. Generally correlated loci will be within the same species. They generally will also be within the same subject. Correlated loci can be correlated via linkage disequilibrium, conserved regions on a haploid, a priori data such as 1000 genomes or the like.

Genomic information can be “phased.” Phased sequences capture unique chromosomal content, including mutations that may differ across chromosome copies. Phased sequencing can, in some instances, distinguish between maternally and paternally inherited alleles.

The term “k-mer” can refers to all the possible subsequences of length k that are contained in a sequence.

A “genome variation map”, can be constructed where individual subject genomes which go into the construction of the map will be merged into the reference genome at the points where it matches the primary sequence, with variations appearing as additional alternate paths along the genome. The resulting map will include multiple forms of genomic variation. A genome variation map can be represented as a graph.

The term “assembly” can be any computational process in which sequence strings produced by a sequencer are merged between one another with the objective to reconstruct the original sequence string, from which the set of all sequence strings were derived.

The term “remote alignment” can be any computational process by which the alignment is divided into a certain predefined number of independent subtasks and for which subtasks can be performed by an independent computer device capable of receiving the sequence strings, of aligning the sequence strings and of transmitting the sequence strings to the appropriate computational device of providing the final whole and complete alignment of all the subtasks.

The term “index” can by any database that is used to optimize the access of data. The database can comprise or consist of keys. These keys can be attributes on which the search on the original database is going to be based.

The term “hash table” can describe a method or structure that can allow for accelerated searching within the index.

The term “reference sequence” can refer to a sequence string composed of the information required to define the molecule at hand. For example, a whole human genome would be a sequence string of nucleotides comprising about 3 billion bases to be compliant for the definition of a human genome. A reference genome (alternately a reference assembly) can be a reference sequence. A reference genome can be a digital nucleic acid sequence database, assembled by as a representative example of a set of related nucleic acids. A reference genome can be, for example, an example of a particular species' or clade's genome. In some instances, a reference genome can comprise alternative paths.

The term “metadata” describes the composition of different types structures added in an ordered manner that can be consistent.

“Raw genetic sequence data” are data obtained from sequencing reactions. Raw genetic sequence data can be text-based, for example it can have a FASTA format. A FASTA format is a text-based format for representing either nucleotide sequences or peptide sequences, in which nucleotides or amino acids are represented using single-letter codes. Raw genetic sequence data can be text-based format for storing both a biological sequence and its corresponding quality scores, for example it can have a FASTQ format. FASTQ format is a text-based format for storing both a biological sequence and its corresponding quality scores. In some instances, the sequence letter and quality score are each encoded with a single ASCII character for brevity. In some instances, raw genetic sequence data can be converted from one format to another using a format converter. In some instances, raw genetic sequence data is called a “read.”

A “sequencing device” is a device that performs a sequencing reaction. Sequencing devices can be used to generate raw genetic sequence data. In some instances, the methods described herein can be performed while the sequencing device is performing the sequencing reaction. For example, as sequence data is generated by the sequencing device those data can be encrypted and aligned while encrypted. In some instances, a sequencing device can output SAM data.

The SAM Format (or “SAM data”) is a text format for storing sequence data in a series of tab delimited ASCII columns. SAM data can be generated as a human readable version of its sister BAM format, which stores the same data in a compressed, indexed, binary form. SAM format data can be output from aligners that read FASTQ files and assign the sequences to a position with respect to a known reference genome. SAM can also be used to archive unaligned sequence data generated directly from sequencing machines. In some instances, SAM data comprises CIGAR strings. The CIGAR string is a sequence of base lengths and the associated operation. They are used to indicate properties, for example which bases align (either a match/mismatch) with the reference, are deleted from the reference, or are insertions that are not in the reference.

The Variant Call Format (VCF) specifies the format of a text file used in bioinformatics for storing gene sequence variations. “VCF data” is data stored in the VCF format. The variant call format stores only the variations need to be stored along with a reference genome.

The General feature format (GFF) stored all of the genetic data, much of which is redundant because it will be shared across the genomes. “GFF data” is data stored in the GFF format.

A “graph alignment” can include the analysis of genomic data using graphs and graph representations. For example, a genome variation map graph can be used to analyze raw sequence data by graph alignment. A graph alignment can be stored in a modified SAM format, here described as DAMN format.

The DAMN Format (or “DAMN data”) is a text format for storing graph aligned sequence data, for example in a series of tab delimited ASCII columns. Reads that are aligned using a graph reference can be written in a SAM format that is compatible with the SAM format for reads aligned against a linear reference. The DAMN format is a format to output reads that are aligned using a graph reference and can include an optional bit flag that is set if the read alignment overlaps a variant, a read tag that characterizes the location of the alignment relative to the reference and/or variant path, and a read tag the indicates which variant the read aligns to. In some cases, the alignment of a read that aligns overlapping with an alternate path is translated back to the linear reference coordinates. In some cases, there is an additional read tag that shows the start of the aligned sequence relative to the coordinate of the variant path. In some cases, there is an additional read that indicates both the start and end of the aligned read relative to the coordinate of the variant path. In some cases, there is an additional read tag that contains alignment scores including, but not limited to, number of matches, mismatches, insertions, deletions, and start position related to the variant path. The read tag can also include alignment scores with respect to the reference path depending on the mapping. In some cases, the start of the alignment indicates a projection to the linear reference path. In some cases, there is an additional read tag indicating if the read could have passed through the alternate path, but it mapped to the reference path. In some cases, there is an additional read tag detailing how many alternate paths the read passed through. In some cases, there is an additional read tag detailing how many alternate paths the read did not pass through, and instead it mapped to the reference path. In some cases, there is an additional read tag detailing if a read starts mapping to a variant path. DAMN format data can be output from aligners that read FASTQ files and assign the sequences to a position with respect to a known graph reference genome. The DAMN format can also be used to archive unaligned sequence data generated directly from sequencing machines. In some instances, DAMN data comprises CIGAR strings. A CIGAR string is a sequence of base lengths and the associated operation. They are used to indicate properties, for example which bases align (either a match/mismatch) with the reference graph, are deleted from the reference graph, or are insertions that are not in the reference graph. Coordinates can be in respect to the linear reference. They can also be anchored in alternate paths, or bubbles, off the linear reference coordinate system. Since the DAMN format is a superset of the SAM format, it is also compatible being converted to the BAM format. All the SAM ASCII columns definitions and order can be preserved in DAMN format, thus facilitating this compatibility.

The term “sequencing”, as used herein, can refer to a method by which the identity of at least 10 consecutive nucleotides (e.g., the identity of at least 20, at least 50, at least 100, at least 200, or at least 500 or more consecutive nucleotides) of a polynucleotide are obtained.

The term “barcode sequence” as used herein, generally refers to a unique sequence of nucleotides that can encode information about an assay. A barcode sequence can encode information relating to the identity of an interrogated allele, identity of a target polynucleotide or genomic locus, identity of a sample, a subject, or any combination thereof. A barcode sequence can be a portion of a primer, a reporter probe, or both. A barcode sequence may be at the 5′-end or 3′-end of an oligonucleotide, or may be located in any region of the oligonucleotide.

Statistical Features

A machine learning approach can learn prediction rules from the associations that it can obtain. Associations can be obtained from features that can be extracted from data and the classes or measurements the machine learning approach is set to predict or describe. This is the “training stage” of the method. For example, prediction rules can be learned from associations between statistical features and genetic features.

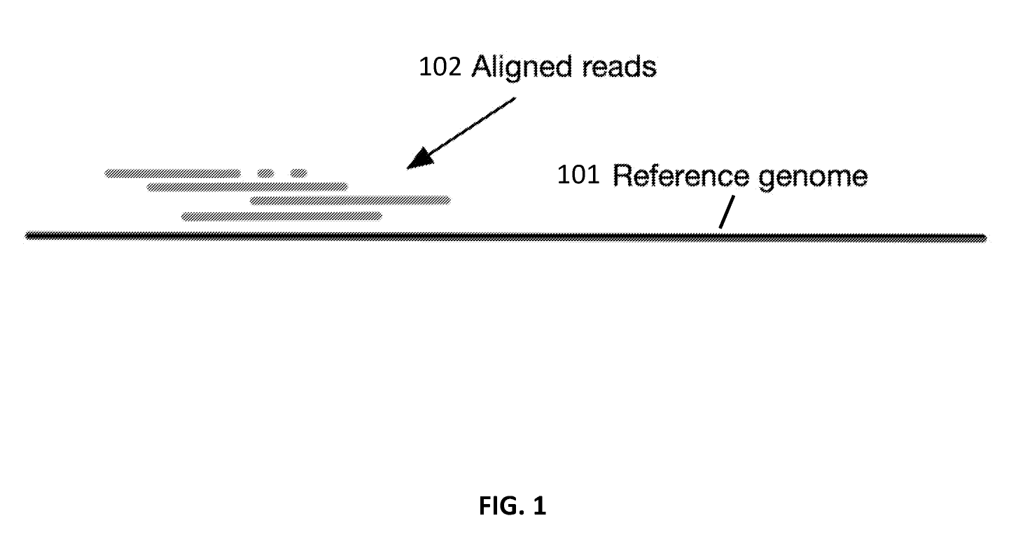

For analysis of nucleotide sequences, the feature source from which the rules are learned are can be files containing alignment data, such as .BAM files. The source of the classes to predict can be files containing sequence data, such as .VCF files. FIG. 1, for example, shows an exemplary schematic of reads 102 aligned to a reference genome or graph 101.

In one example, .BAM files were generated from .FASTQ information provided from the 1KGenomes project. The training samples came from the Phase III stage of the project: NA12827, NA12828, NA12829, NA12830, NA12842, NA12843, NA12872, NA12873 and NA12874. All are from European descent and were generated in 2011 and 2013. As for the .VCF files from which variant types were extracted are also provided by the 1KGenomes project. The .VCF information comes from multiple runs, which can make it a challenging experimental design to test.

The statistical features that can be analyzed (e.g., those obtained from the .BAM files from aligned regions from a graph aligner that have no alternative paths or bubbles) can include but are not limited to the statistical features shown in List 1:

List 1. Statistical Features.

1. % A

2. % C

3. % G

4. % T

5. % AT

6. % GC

7. CIGAR: I (# of insertions)

8. CIGAR: D (# of deletions)

9. CIGAR: S (# of soft clips)

10. CIGAR: H (# of hard clips)

11. Mean depth

12. Mean MAPQ score

13. # of reads that insert size is greater than a the % q0 quantile

14. # of reads that insert size is smaller than a the % q0 quantile

15. # of reads on the positive strand

16. # of reads on the negative strand

17. percent of reads with a correct orientation

18. # of reads with the 0 x 1 BAM flag

19. # of reads with the 0 x 2 BAM flag

20. # of reads with the 0 x 4 BAM flag

21. # of reads with the 0 x 8 BAM flag

22. # of reads with the 0 x 20 BAM flag

23. # of reads with the 0 x 40 BAM flag

24. # of reads with the 0 x 80 BAM flag

25. # of reads with the 0 x 100 BAM flag

26. # of reads with the 0 x 200 BAM flag

27. # of reads with the 0 x 400 BAM flag

28. # of reads with the 0 x 800 BAM flag

Many statistical features (such as the features in List 1, with the exception of the first six which are already expressed as percentages), may be normalized to have values between 0 and 1 (e.g., a percentage). This can provide numerical stability when running the machine learning procedures. Table 1 provides explanations of the different BAM flags. Use of comparable statistical features with file formats other than BAM are contemplated.

TABLE 1
Explanation of BAM flags.

| BAM Flag Bit | Description |
|---|---|
| 0x1 | template having multiple segments in sequencing |
| 0x2 | each segment properly aligned according to the aligner |
| 0x4 | segment unmapped |
| 0x8 | next segment in the template unmapped |
| 0x10 | SEQ being reverse complemented |
| 0x20 | SEQ of the next segment in the template being reversed |
| 0x40 | the first segment in the template |
| 0x80 | the last segment in the template |
| 0x100 | secondary alignment |
| 0x200 | not passing quality controls |
| 0x400 | PCR or optical duplicate |
| 0x800 | supplementary alignment |

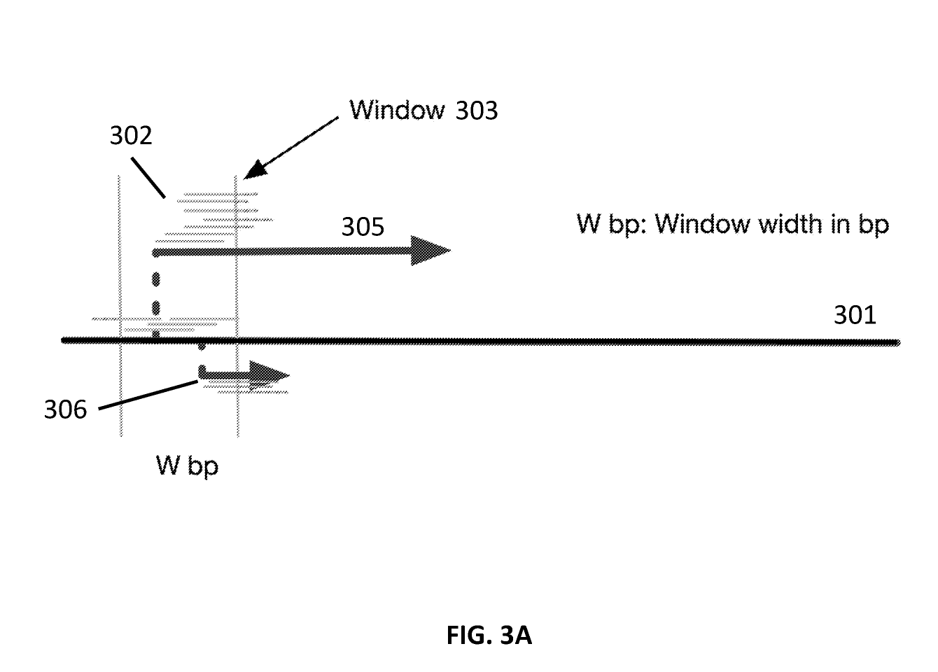

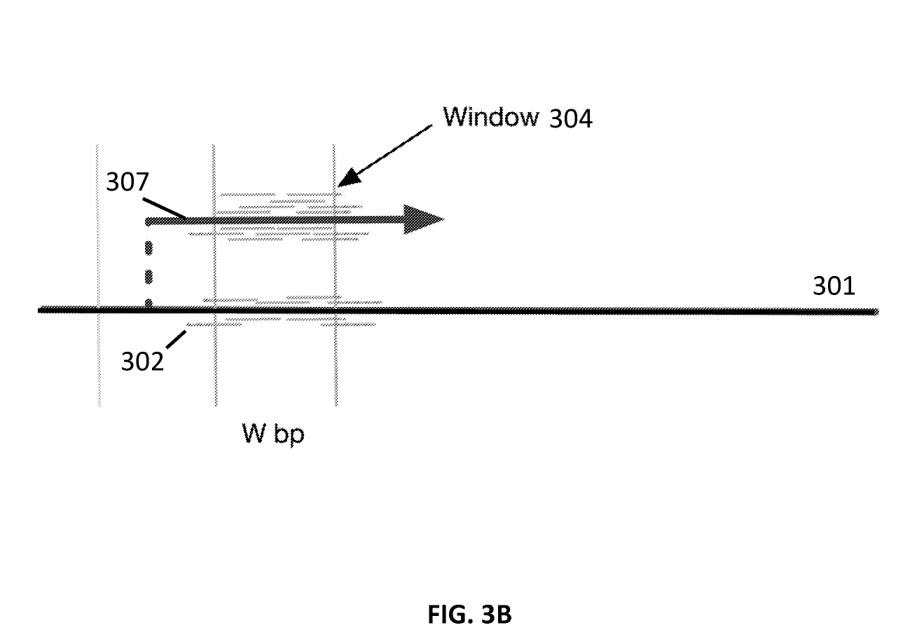

Some aligned regions can have bubbles or multiple paths, such as when using a graph alignment. For feature extraction for aligned regions that contain bubbles or multiple paths, additional statistical features can be analyzed. To make the extraction of information be useful for the machine learning method chosen, there can be a need to define the total number features to be considered beforehand. In such cases, B can be defined as the maximum number of paths or bubbles that can be within in a window of length w. FIG. 3A and FIG. 3B, for example, show an exemplary schematic of reads 302 aligned to a reference 301 in a region with bubbles or multiple paths. Reads considered within the first window 303 consider the start of the bubbles (see FIG. 3A), while reads considered within the second window 304 consider sections within the bubble (see FIG. 3B). Additional statistical features that can be considered (e.g., from a graph aligner) can include but are not limited to those shown in List 2. Some features in List 2 may be normalized to have values between 0 and 1 (e.g., a percentage).

List 2. Additional statistical features.

1. # of paths or bubbles that fall within the window of width w

2. # of starts of paths or bubbles that fall within the window of width w

3. # of ends of path or bubbles that fall within the window of width w

4. # of complete sections of paths or bubbles that fall within the window of width w

5. Mean depth for each path or bubble that fall within the window of width w

6. The significance of each path or bubble that falls within the window of width w

7. The portion of the total length of each path of bubble that falls within the window of width w

8. All the information from the VCF file for each path or bubble that fall within the window of width w

### Genetic Features

The techniques described herein can be performed to assess, detect, predict, characterize, or otherwise analyze genetic features.

Genetic features can include variants, markers, traits, and other features. Variants can include structural variants, such as deletions, insertions, and inversions. A variant can be an alteration in the normal sequence of a nucleic acid sequence (e.g., a gene). In some instances, a genotype and corresponding phenotype is associated with a variant. In other instances, there is no known function of a variant. A variant can be a SNP. A variant can be a SNV. A variant can be an insertion of a plurality of nucleotides. A variant can be a deletion of a plurality of nucleotides. A variant can be a mutation. A variant can be a copy number variation (CNV). A variant can be a structural variant (SV). Structural variants can be classified according to their length: short: (6 bp≤SV<50 bp), medium: (50 bp≤SV<500 bp), and large: (500 bp≤SV). A variant can be a nucleic acid deviation between two or more individuals in a population. Markers can include individual subject markers, taxonomic markers (e.g., clade markers, strain markers, sub-strain markers, species markers), resistance markers (e.g., antibiotic resistance markers), susceptibility markers (e.g., antibiotic susceptibility markers), pathogenicity markers, virulence markers, and other trait markers.

Genetic features can include any genome, genotype, haplotype, chromatin, chromosome, chromosome locus, chromosomal material, deoxyribonucleic acid (DNA), allele, gene, gene cluster, gene locus, genetic polymorphism, genetic mutation, genetic mutation rate, nucleotide, nucleotide base pair, single nucleotide polymorphism (SNP), restriction fragment length polymorphism (RFLP), variable tandem repeat (VTR), copy number variant (CNV), microsatellite sequence, genetic marker, sequence marker, flanking region, sequence tagged site (STS), plasmid, transcription unit, transcription product, gene expression level, genetic expression (e.g., transcription) state, ribonucleic acid (RNA), complementary DNA (cDNA), conserved region, and pathogenicity island, including the nucleotide sequence and encoded amino acid sequence associated with any of the above. An epigenetic feature is any feature of genetic material—all genomic, vector and plasmid DNA and chromatin—that affects gene expression in a manner that is heritable during somatic cell divisions and sometimes heritable in germline transmission, but that is non-mutational to the DNA sequence, including but not limited to methylation of DNA nucleotides and acetylation of chromatin-associated histone proteins. As used herein, therefore, genetic sequence data can include, without limitation, nucleotide sequences, deoxyribonucleic acid (DNA) sequences, and ribonucleic acid (RNA) sequences.

Genetic features can include subject-specific features. A subject specific feature can refer to any feature or attribute that is capable of distinguishing one subject from another. In some cases, a subject-specific feature is a genetic feature. The genetic feature, as described above, can be present on a nucleic acid isolated from a subject. In some cases, a subject-specific feature can relate to a feature or features that distinguish a set of functions. Subject-specific features can include a single gene, a plurality of genes, or genomic regions with known epigenomic functions such as promoter regions.

The term “mutation”, as used herein, generally refers to a change of the nucleotide sequence of a genome. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms, multiple nucleotide polymorphisms, insertions (e.g., insertion of one or more nucleotides at a locus), multiple nucleotide changes, deletions (e.g., deletion of one or more nucleotides at a locus), and inversions (e.g., reversal of a sequence of one or more nucleotides).

The term “locus”, as used herein, can refer to a location of a gene, nucleotide, or sequence on a chromosome. An “allele” of a locus, as used herein, can refer to an alternative form of a nucleotide or sequence at the locus. A “wild-type allele” generally refers to an allele that has the highest frequency in a population of subjects. A “wild-type” allele generally is not associated with a disease. A “mutant allele” generally refers to an allele that has a lower frequency that a “wild-type allele” and can be associated with a disease. A “mutant allele” may not have to be associated with a disease. The term “interrogated allele” generally refers to the allele that an assay is designed to detect.

The term “single nucleotide polymorphism”, or “SNP”, as used herein, generally refers to a type of genomic sequence variation resulting from a single nucleotide substitution within a sequence. “SNP alleles” or “alleles of a SNP” generally refer to alternative forms of the SNP at particular locus. The term “interrogated SNP allele” generally refers to the SNP allele that an assay is designed to detect.

### Machine Learning and Algorithm Training

To perform the “training stage” following a machine learning paradigm, feature information can be extracted and the classes to be predicted can be associated to the features. Training directly impacts the final performances of the learned rules. Accordingly, high quality “training data” can be important.

### Feature Extraction

Feature extraction can be performed using a moving (or sliding) window method. For example, a moving window of fixed width w base pairs (bp) can be used throughout the alignment. In some cases, a variable length window can be used.

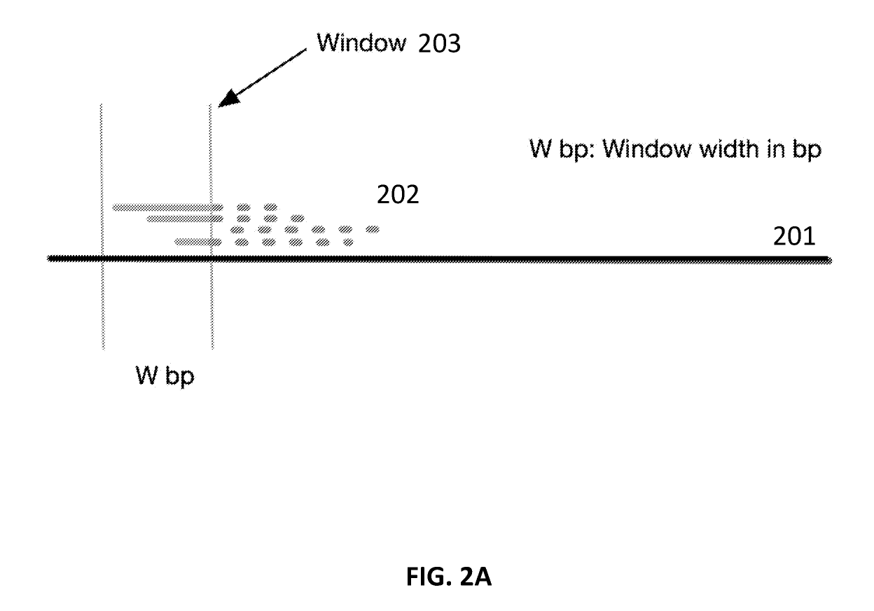

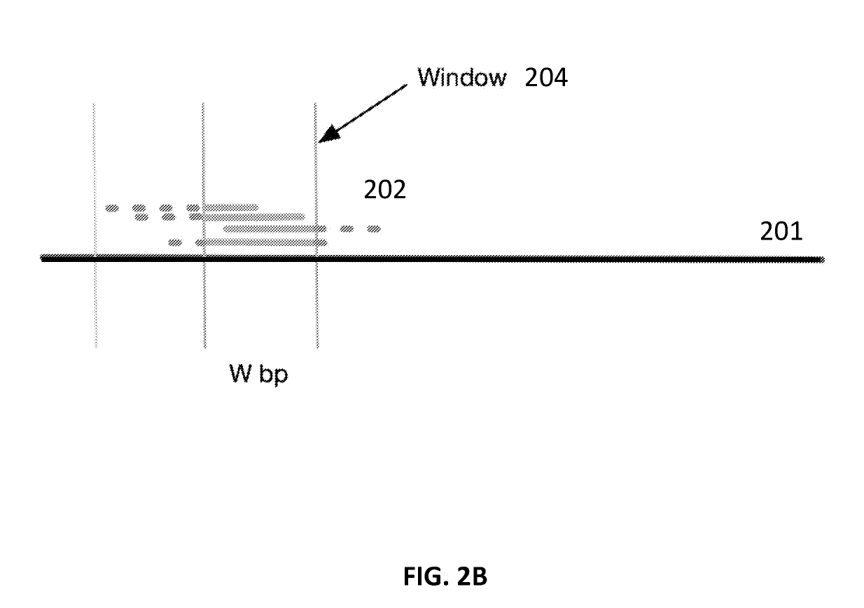

In an example, W is defined as the window length. Based on the window length of W, the section of the reads that fall within the window can be identified. From these sections of reads, the features can be obtained. For example, FIG. 2A shows only the solid-line sections of the reads 202 (aligned to a reference 201) have their limits within a window 203 of length W. Accordingly, the selected statistical features can be determined for only the solid-line section of the reads and associated to that particular window. FIG. 2B shows the subsequent window 204, with only the solid-line sections of the reads having their limits within the window. The selected statistical features can be determined only for the solid-line section of the reads and associated with that second window. Following this iterative procedure of sequentially sliding the window can extract all the relevant information from the alignment. This information can be registered into matrices, for example of dimensions nᵢ×m, where nᵢ is the number of total windows within chromosome i and m is the total number of statistical features (e.g., 28 from List 1) such as those described herein.

Analysis can be performed on unsorted files (e.g., unsorted .BAM, .SAM, or .DAMN files). This provides an advantage over previously reported methods that required or were improved by use of a sorted file, which can require additional computation to prepare. To analyze an unsorted file, windows can created and distributed along a reference genome or graph reference. Aligned sequences can accumulate in the window via knowing the alignment position. From that information, any other statistics can be determined from the read, the alignment, and accumulated in the window. Since the window spans a width, the number of windows goes as length of genome divided by the width of the window. In an example, windows of width 100 along the human genome, with 3×10⁹ bases, yields 3×10⁷ windows. All window indices can be stored in, at most 115 MB, assuming each index is an unsigned integer. Further, the window is fully defined with a start position and an end position. A data structure can be created with the statistical features and maintain a data pointer in the window towards this data structure. To populate the windows, an unsorted SAM, BAM, or DAMN format can be read sequentially. Given the starting position of the read and the CIGAR score, it immediately can be determined to which window that read belongs. In this way, all unsorted reads are placed in sorted windows, with the statistics accumulating as the sequential unsorted files are read.

Statistical features and other characteristics of a genetic sequence can be obtained by a feature obtention module (e.g., via a moving window method). Statistical feature information can be passed to a genetic feature (e.g., structural variant) breakpoint classification module. Separately, candidates for genetic feature (e.g., structural variant) breakpoints can be identified, for example based on coverage and orientation of sequence. Breakpoint candidates can also be passed to the genetic feature breakpoint classification module to assist in the classification. The use of breakpoint candidates in genetic feature breakpoint classification can reduce or minimize false positives (increase or maximize specificity).

The genetic feature breakpoint classification module can then classify breakpoints, producing information including but not limited to insertion ends, deletion ends, and neutral copy number ends (e.g., for structural variants). This information can be passed to a genetic feature breakpoint merging module.

The genetic feature breakpoint merging module can receive one or more categories of information about genetic features and merge them into a unified identification. For example, the merging module can unify information about genetic feature (e.g., structural variant) insertions, deletions, and copy number variations. The merging module can receive information from a genetic feature breakpoint classification module as discussed above. The merging module can also receive information such as prior population information and prior reference information about genetic features, merge this information into the analysis. Inclusion of this additional information, such as genetic feature prior reference information, can reduce or minimize false negatives (increase or maximize sensitivity).

Identification and compilation of features can be conducted on a graph reference. List 2, for example, provides non-limiting examples of statistical features than can be employed when analyzing with a graph reference. Such features can be obtained from reading a graph formatted file, such as a .DAMN file. Use of a graph format can provide more detail and improve accuracy in determining breakpoints of genetic features such as variants (e.g., structural variants) and markers (e.g., individual subject markers, clade markers, strain markers, sub-strain markers, species markers).

In some cases, graph alignment files can be large. To more efficiently process such files, statistical features can be used to provide an initial analysis. Such an analysis can be used to identify regions that need additional computation or further analysis.

The total number of statistical features analyzed can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more. The total number of statistical features analyzed can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more.

Window length can be at least about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, 1000, or more base pairs. Window length can be at most about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, or 1000 base pairs. Window length can be about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, 1000, or more base pairs. Window length can be at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, 1000, or more base pairs. Window length can be at most 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, or 1000 base pairs. Window length can be 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, 1000, or more base pairs.

### Machine Learning Techniques

A variety of machine learning techniques and statistical methods can be employed with the techniques disclosed herein. See, for example, Michaelson and Sebat, “forestSV: structural variant discovery through statistical learning,” Nature Methods, 9(8): 819-822, 2012. Statistical methods can include but are not limited to penalized logistic regression, prediction analysis of microarrays (PAM), shrunken centroid-based methods, support vector machine analysis, and regularized linear discriminant analysis. Machine learning techniques can include but are not limited to bagging procedures, boosting procedures, random forest algorithms, neural networks, and any combination thereof. In some cases, a simple linear regression model is sufficient for a particular analysis.

Machine learning techniques can be trained using a set of samples, such as a sample cohort. The sample cohort can comprise at least about 5, 10, 20, 30, 40, 50, 60, 70, 80, 90, 100, 150, 200, 250, 300, 350, 400, 450, 500, 600, 700, 800, 900, 1000, 2000, 3000, 4000, 5000 or more independent samples. The sample cohort can comprise at least about 100 independent samples. The sample cohort can comprise at least about 200 independent samples. The sample cohort can comprise between about 100 and about 500 independent samples. The independent samples can be from subjects having been diagnosed with a disease, such as cancer, from healthy subjects, or any combination thereof

The sample cohort can comprise samples from at least about 5, 10, 20, 30, 40, 50, 60, 70, 80, 90, 100, 150, 200, 250, 300, 350, 400, 450, 500, 600, 700, 800, 900, 1000 or more different individuals. The sample cohort can comprise samples from at least about 100 different individuals. The sample cohort can comprise samples from at least about 200 different individuals. The different individuals can be individuals having been diagnosed with a disease, such as cancer, healthy individuals, or any combination thereof.

The sample cohort can comprise samples obtained from individuals living in at least 1, 2, 3, 4, 5, 6, 67, 8, 9, 10, 15, 20, 25, 30, 35, 40, 45, 50, 55, 60, 65, 70, 75, or 80 different geographical locations (e.g., sites spread out across a nation, such as the United States, across a continent, or across the world). Geographical locations include, but are not limited to, test centers, medical facilities, medical offices, post office addresses, cities, counties, states, nations, or continents. In some cases, a machine learning technique that is trained using sample cohorts from the United States may need to be re-trained for use on sample cohorts from other geographical regions (e.g., India, Asia, Europe, Africa, etc.).

Performance

Identification or classification of a genetic feature by a machine learning technique can be performed at high accuracy. A genetic feature can be identified or classified with an accuracy of at least about 50%, 60%, 70%, 75%, 80%, 85%, 90%, 95%, 96%, 97%, 98%, 99%, 99.9%, 99.99%, 99.999% or more. A genetic feature can be identified or classified with an accuracy of at least 70%. A genetic feature can be identified or classified with an accuracy of at least 80%. A genetic feature can be identified or classified with an accuracy of at least 90%.

Identification or classification of a genetic feature by a machine learning technique can be performed at high specificity. A genetic feature can be identified or classified with a specificity of at least about 50%, 60%, 70%, 75%, 80%, 85%, 90%, 95%, 96%, 97%, 98%, 99%, 99.9%, 99.99%, 99.999% or more. A genetic feature can be identified or classified with a specificity of at least 70%. A genetic feature can be identified or classified with a specificity of at least 80%. A genetic feature can be identified or classified with a specificity of at least 90%.

Identification or classification of a genetic feature by a machine learning technique can be performed at high sensitivity. A genetic feature can be identified or classified with a sensitivity of at least about 50%, 60%, 70%, 75%, 80%, 85%, 90%, 95%, 96%, 97%, 98%, 99%, 99.9%, 99.99%, 99.999% or more. A genetic feature can be identified or classified with a sensitivity of at least 70%. A genetic feature can be identified or classified with a sensitivity of at least 80%. A genetic feature can be identified or classified with a sensitivity of at least 90%.

Use of the machine learning techniques of the present disclosure can improve the functioning of the computer systems on which they are implemented. For example, the machine learning techniques can reduce the processing time for a given analysis by at least about 5%, 10%, 15%, 20%, 25%, 30%, 35%, 40%, 45%, 50%, 55%, 60%, 65%, 70%, 75%, 80%, 85%, 90%, 95%, or more. The machine learning techniques can reduce the memory requirements for a given analysis by at least about 5%, 10%, 15%, 20%, 25%, 30%, 35%, 40%, 45%, 50%, 55%, 60%, 65%, 70%, 75%, 80%, 85%, 90%, 95%, or more.

Use of the machine learning techniques of the present disclosure can enable conducting analyses that were previously not possible. For example, certain genetic features can be detected from sequence information (e.g., aligned reads) that would not be detectable from such information without the methods of the present disclosure.

Computer Control Systems

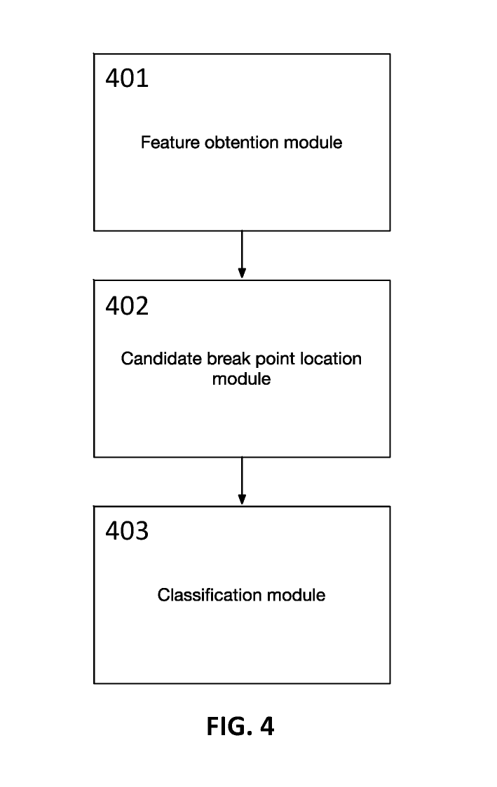

The present disclosure provides computer control systems that are programmed to implement methods of the disclosure. FIG. 4 shows an exemplary schematic of a system configured to implement the methods provided herein, comprising a feature obtention module 401, a candidate break point location module 402, and a classification module 403. The feature obtention module can obtain statistical features from a sequence. Some of these statistical features (e.g., coverage, orientation) can be used by the candidate break point location module to identify candidate break points for structural variants and other genetic features. Statistical features, and in some cases break point candidate identifications, then can be used by the classification module to classify various genetic features.

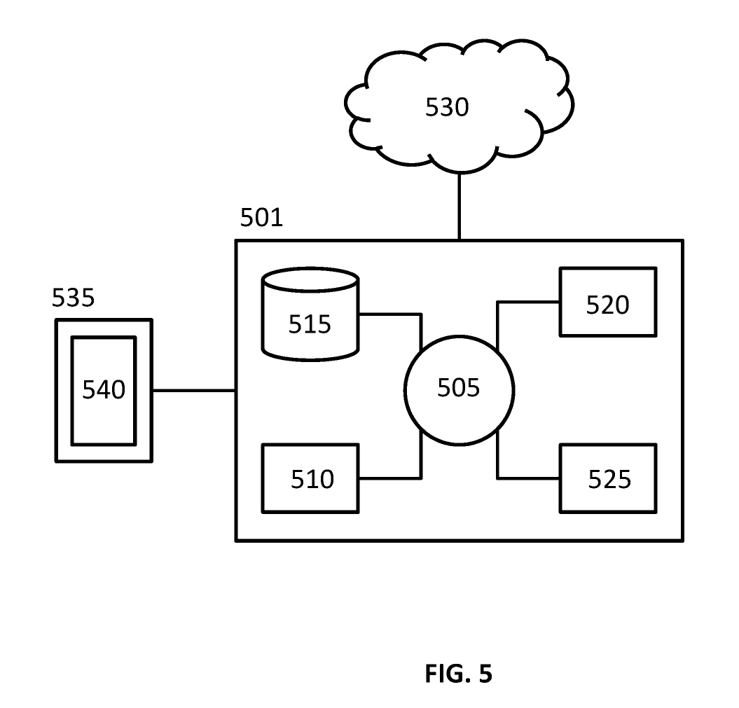

FIG. 5 shows a computer system 501 that is programmed or otherwise configured to implement the methods provided herein. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software.

During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD- ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, an output or readout of the trained algorithm. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 505.

### Subjects and Samples

The techniques of the present disclosure can be practiced on a wide variety of types of subjects and samples.

The term “subject”, as used herein, generally refers to a specific source of genetic materials. The subject can be a biological entity. The biological entity can be a plant, animal, or microorganism, including, e.g., bacteria, viruses, fungi, and protozoa. The subject can be an organ, tissue, or cell. A subject can be obtained in vivo or cultured in vitro. The subject can be a cell line. The subject can be propagated in culture. The subject can be disease cells. The subject can be cancer cells. The subject can be a mammal. The mammal can be a human. The subject can mean an individual representation of the specific source of genetic material (e.g., the subject can be a particular individual human or a particular bacterial strain). Alternatively, the subject can be a general representation of a kind of specific source of genetic materials, e.g. the subject can be any and all members of a single species or clade. The subject can also be a portion of a genome, for example if the sample does not contain a full genome.

A “sample” or “nucleic acid sample” can refer to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA. The nucleic acids in a nucleic acid sample can serve as templates for extension of a hybridized primer. In some cases, the biological sample is a liquid sample. The liquid sample can be, for example, whole blood, plasma, serum, ascites, semen, cerebrospinal fluid, sweat, urine, tears, saliva, buccal sample, cavity rinse, or organ rinse. The liquid sample can be an essentially cell-free liquid sample (e.g., plasma, serum, sweat, urine, tears, etc.). In other cases, the biological sample is a solid biological sample, e.g., feces, hair, nail, or tissue biopsy, e.g., a tumor biopsy. A sample can also comprise in vitro cell culture constituents (including but not limited to conditioned medium resulting from the growth of cells in cell culture medium, recombinant cells and cell components). A sample can comprise or be derived from cancer cells. A sample can comprise a microbiome.

A “complex sample” as used herein refers to a sample that includes two or more subjects or that includes material (e.g., nucleic acids) from two or more subjects. A complex sample can comprise genetic material from two or more subjects. A complex sample can comprise nucleic acid molecules from two or more subjects. A complex sample can comprise nucleic acids from two or more strains of bacteria, viruses, fungi and the like. A complex sample can comprise two or more resolvable subjects (i.e., two or more subjects that are distinguishable from one another). In some cases, complex samples can be obtained from the environment. For example, a complex sample can be an air sample, a soil or dirt sample or a water sample (e.g., river, lake, ocean, wastewater, etc.). Environmental samples can comprise one or more species, subspecies, strains, sub-strains, or clades of bacteria, viruses, protozoans, algae, fungi and the like.

“Nucleotides” can be biological molecules that can form nucleic acids. Nucleotides can have moieties that contain not only the known purine and pyrimidine bases, but also other heterocyclic bases that have been modified. Such modifications include methylated purines or pyrimidines, acylated purines or pyrimidines, alkylated riboses, or other heterocycles. In addition, the term “nucleotide” includes those moieties that contain hapten, biotin, or fluorescent labels and can contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the like.

“Nucleotides” can also include locked nucleic acids (LNA) or bridged nucleic acids (BNA). BNA and LNA generally refer to modified ribonucleotides wherein the ribose moiety is modified with a bridge connecting the 2′ oxygen and 4′ carbon. Generally, the bridge “locks” the ribose in the 3′-endo (North) conformation, which is often found in the A-form duplexes. The term “locked nucleic acid” (LNA) generally refers to a class of BNAs, where the ribose ring is “locked” with a methylene bridge connecting the 2′-O atom with the 4′-C atom. LNA nucleosides containing the six common nucleobases (T, C, G, A, U and mC) that appear in DNA and RNA are able to form base-pairs with their complementary nucleosides according to the standard Watson-Crick base pairing rules. Accordingly, BNA and LNA nucleotides can be mixed with DNA or RNA bases in an oligonucleotide whenever desired. The locked ribose conformation enhances base stacking and backbone pre-organization. Base stacking and backbone pre-organization can give rise to an increased thermal stability (e.g., increased Tm) and discriminative power of duplexes. LNA can discriminate single base mismatches under conditions not possible with other nucleic acids.

The terms “polynucleotides”, “nucleic acid”, “nucleotides” and “oligonucleotides” can be used interchangeably. They can refer to a polymeric form of nucleotides of any length, either deoxyribonucleotides or ribonucleotides, or analogs thereof. Polynucleotides can have any three-dimensional structure, and can perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component.

The term “target polynucleotide” or “target nucleic acid” as used herein, generally refers to a polynucleotide of interest under study. In certain cases, a target polynucleotide contains one or more sequences that are of interest and under study. A target polynucleotide can comprise, for example, a genomic sequence. The target polynucleotide can comprise a target sequence whose presence, amount, and/or nucleotide sequence, or changes in these, are desired to be determined. A target polynucleotide can comprise non-coding regions of a genome.

The term “genome” can refer to the genetic complement of a biological organism, and the terms “genomic data” and “genomic data set” include sequence information of chromosomes, genes, or DNA of the biological organism.

The term “genomic data,” as used herein, refers to data that can be one or more of the following: the genome or exome sequence of one or more, or any combination or mixture of one or more, mitochondria, cells, including eggs and sperm, tissues, neoplasms, tumors, organs, organisms, microorganisms, viruses, individuals, or cell free DNA, and further including, but not limited to, nucleic acid sequence information, genotype information, gene expression information, genetic data, epigenetic information including DNA methylation, acetylation or similar DNA modification data, RNA transcription, splicing, editing or processing information, or medical, health or phenotypic data, or nutritional, dietary or environmental condition or exposure information or other attribute data of any microorganism, virus, cell, tissue, neoplasm, tumor, organ, organ system, cell-free sample (e.g. serum or media), individual or group of samples or individuals. Accordingly, the term “genornic sequence,” as used herein, refers to a sequence that occurs in a genome. Because RNAs are transcribed from a genome, this term encompasses sequences that exist in the nuclear genome of an organism, as well as sequences that are present in a cDNA copy of an RNA (e.g., an mRNA) transcribed from such a genome. “Genomic sequence” can also be a sequence that occurs on the cytoplasm or in the mitochondria

The terms “determining,” “measuring,” “evaluating,” “assessing,” “assaying,” and “analyzing” can be used interchangeably herein to refer to any form of measurement, and can include determining if an element is present or not. These terms can include both quantitative and/or qualitative determinations. Assessing can be relative or absolute. “Assessing the presence of” can include determining the amount of something present, as well as determining whether it is present or absent.

The term “genomic fragment”, as used herein, can refer to a region of a genome, e.g., an animal or plant genome such as the genome of a human, monkey, rat, fish or insect or plant. A genomic fragment may or may not be adaptor ligated. A genomic fragment can be adaptor ligated (in which case it has an adaptor ligated to one or both ends of the fragment, to at least the 5′ end of a molecule), or non-adaptor ligated.

### Sequencing Platforms

The techniques of the present disclosure can be used on sequencing data from a variety of sequencing platforms, including next-generation sequencing platforms. Sequence data can be from partial sequencing or complete sequencing of DNA (e.g., DNA fragments) in a sample.

The next-generation sequencing platform can be a commercially available platform. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer, and are described in Gudmundsson et al (Nat. Genet. 2009 41: 1122-6), Out et al (Hum. Mutat. 2009 30: 1703-12) and Turner (Nat. Methods 2009 6: 315-6), U.S. Patent Application Pub nos. US20080160580 and US20080286795, U.S. Pat. Nos. 6,306,597, 7,115,400, and 7,232,656. 454 Life Science platforms include, e.g., the GS Flex and GS Junior, and are described in U.S. Pat. No. 7,323,305. Platforms from Helicos Biosciences include the True Single Molecule Sequencing platform. Platforms for ion semiconductor sequencing include, e.g., the Ion Torrent Personal Genome Machine (PGM) and are described in U.S. Pat. No. 7,948,015. Platforms for pyrosequencing include the GS Flex 454 system and are described in U.S. Pat. Nos. 7,211,390; 7,244,559; 7,264,929. Platforms and methods for sequencing by ligation include, e.g., the SOLiD sequencing platform and are described in U.S. Pat. No. 5,750,341. Platforms for single-molecule sequencing include the SMRT system from Pacific Bioscience and the Helicos True Single Molecule Sequencing platform.

While the automated Sanger method is considered as a ‘first generation’ technology, Sanger sequencing including the automated Sanger sequencing, can also be employed by the method of the invention. Additional sequencing methods that comprise the use of developing nucleic acid imaging technologies e.g. atomic force microscopy (AFM) or transmission electron microscopy (TEM), are also encompassed by the method of the invention. Exemplary sequencing technologies are described below.

The next generation sequencing technology can utilize the Ion Torrent sequencing platform, which pairs semiconductor technology with a sequencing chemistry to directly translate chemically encoded information (A, C, G, T) into digital information (0, 1) on a semiconductor chip. Without wishing to be bound by theory, when a nucleotide is incorporated into a strand of DNA by a polymerase, a hydrogen ion is released as a byproduct. The Ion Torrent platform detects the release of the hydrogen atom as a change in pH. A detected change in pH can be used to indicate nucleotide incorporation. The Ion Torrent platform comprises a high-density array of micro-machined wells to perform this biochemical process in a massively parallel way. Each well holds a different library member, which may be clonally amplified. Beneath the wells is an ion-sensitive layer and beneath that an ion sensor. The platform sequentially floods the array with one nucleotide after another. When a nucleotide, for example a C, is added to a DNA template and is then incorporated into a strand of DNA, a hydrogen ion will be released. The charge from that ion will change the pH of the solution, which can be identified by Ion Torrent's ion sensor. If the nucleotide is not incorporated, no voltage change will be recorded and no base will be called. If there are two identical bases on the DNA strand, the voltage will be double, and the chip will record two identical bases called. Direct identification allows recordation of nucleotide incorporation in seconds. Library preparation for the Ion Torrent platform generally involves ligation of two distinct adaptors at both ends of a DNA fragment.

The next generation sequencing technology can utilize an Illumina sequencing platform, which generally employs cluster amplification of library members onto a flow cell and a sequencing-by-synthesis approach. Cluster-amplified library members are subjected to repeated cycles of polymerase-directed single base extension. Single-base extension can involve incorporation of reversible-terminator dNTPs, each dNTP labeled with a different removable fluorophore. The reversible-terminator dNTPs are generally 3′ modified to prevent further extension by the polymerase. After incorporation, the incorporated nucleotide can be identified by fluorescence imaging. Following fluorescence imaging, the fluorophore can be removed and the 3′ modification can be removed resulting in a 3′ hydroxyl group, thereby allowing another cycle of single base extension. Library preparation for the Illumina platform generally involves ligation of two distinct adaptors at both ends of a DNA fragment.

The next generation sequencing technology that is used in the method of the invention can be the Helicos True Single Molecule Sequencing (tSMS), which can employ sequencing-by-synthesis technology. In the tSMS technique, a polyA adaptor can be ligated to the 3′end of DNA fragments. The adapted fragments can be hybridized to poly-T oligonucleotides immobilized on the tSMS flow cell. The library members can be immobilized onto the flow cell at a density of about 100 million templates/cm2. The flow cell can be then loaded into an instrument, e.g., HeliScope™ sequencer, and a laser can illuminate the surface of the flow cell, revealing the position of each template. A CCD camera can map the position of the templates on the flow cell surface. The library members can be subjected to repeated cycles of polymerase-directed single base extension. The sequencing reaction begins by introducing a DNA polymerase and a fluorescently labeled nucleotide. The polymerase can incorporate the labeled nucleotides to the primer in a template directed manner. The polymerase and unincorporated nucleotides can be removed. The templates that have directed incorporation of the fluorescently labeled nucleotide can be discerned by imaging the flow cell surface. After imaging, a cleavage step can remove the fluorescent label, and the process can be repeated with other fluorescently labeled nucleotides until a desired read length is achieved. Sequence information can be collected with each nucleotide addition step.

The next generation sequencing technology can utilize a 454 sequencing platform (Roche) (e.g. as described in Margulies, M. et al. Nature 437: 376-380 [2005]). 454 sequencing generally involves two steps. In a first step, DNA can be sheared into fragments. The fragments can be blunt-ended. Oligonucleotide adaptors can be ligated to the ends of the fragments. The adaptors generally serve as primers for amplification and sequencing of the fragments. At least one adaptor can comprise a capture reagent, e.g., a biotin. The fragments can be attached to DNA capture beads, e.g., streptavidin-coated beads. The fragments attached to the beads can be PCR amplified within droplets of an oil-water emulsion, resulting in multiple copies of clonally amplified DNA fragments on each bead. In a second step, the beads can be captured in wells, which can be pico-liter sized. Pyrosequencing can be performed on each DNA fragment in parallel. Pyrosequencing generally detects release of pyrophosphate (PPi) upon nucleotide incorporation. PPi can be converted to ATP by ATP sulfurylase in the presence of adenosine 5′ phosphosulfate. Luciferase can use ATP to convert luciferin to oxyluciferin, thereby generating a light signal that is detected. A detected light signal can be used to identify the incorporated nucleotide.

The next generation sequencing technology can utilize a SOLiD™ technology (Applied Biosystems). The SOLiD platform generally utilizes a sequencing-by-ligation approach. Library preparation for use with a SOLiD platform generally comprises ligation of adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured. Beads can be enriched for beads with extended templates. Templates on the selected beads can be subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide can be removed and the process can then be repeated.

The next generation sequencing technology can utilize a single molecule, real-time (SMRT™) sequencing platform (Pacific Biosciences). In SMRT sequencing, the continuous incorporation of dye-labeled nucleotides can be imaged during DNA synthesis. Single DNA polymerase molecules can be attached to the bottom surface of individual zero-mode wavelength identifiers (ZMW identifiers) that obtain sequence information while phospholinked nucleotides are being incorporated into the growing primer strand. A ZMW generally refers to a confinement structure which enables observation of incorporation of a single nucleotide by DNA polymerase against a background of fluorescent nucleotides that rapidly diffuse in an out of the ZMW on a microsecond scale. By contrast, incorporation of a nucleotide generally occurs on a milliseconds timescale. During this time, the fluorescent label can be excited to produce a fluorescent signal, which is detected. Detection of the fluorescent signal can be used to generate sequence information. The fluorophore can then be removed, and the process repeated. Library preparation for the SMRT platform generally involves ligation of hairpin adaptors to the ends of DNA fragments.

The next generation sequencing technology can utilize nanopore sequencing (e.g. as described in Soni G V and Meller A. Clin Chem 53: 1996-2001 [2007]). Nanopore sequencing DNA analysis techniques are being industrially developed by a number of companies, including Oxford Nanopore Technologies (Oxford, United Kingdom). Nanopore sequencing is a single-molecule sequencing technology whereby a single molecule of DNA is sequenced directly as it passes through a nanopore. A nanopore can be a small hole, of the order of 1 nanometer in diameter. Immersion of a nanopore in a conducting fluid and application of a potential (voltage) across can result in a slight electrical current due to conduction of ions through the nanopore. The amount of current which flows is sensitive to the size and shape of the nanopore and to occlusion by, e.g., a DNA molecule. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence.

The next generation sequencing technology can utilize a chemical-sensitive field effect transistor (chemFET) array (e.g., as described in U.S. Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

The next generation sequencing technology can utilize transmission electron microscopy (TEM). The method, termed Individual Molecule Placement Rapid Nano Transfer (IMPRNT), generally comprises single atom resolution transmission electron microscope imaging of high-molecular weight (150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA. The method is further described in PCT patent publication WO 2009/046445. The method allows for sequencing complete human genomes in less than ten minutes.

The method can utilize sequencing by hybridization (SBH). SBH generally comprises contacting a plurality of polynucleotide sequences with a plurality of polynucleotide probes, wherein each of the plurality of polynucleotide probes can be optionally tethered to a substrate. The substrate might be flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be identified and used to identify the plurality of polynucleotide sequences within the sample.

The length of the sequence read can vary depending on the particular sequencing technology utilized. NGS platforms can provide sequence reads that vary in size from tens to hundreds, or thousands of base pairs, or even tens or hundreds of thousands of base pairs. In some embodiments of the method described herein, the sequence reads are about 20 bases long, about 25 bases long, about 30 bases long, about 35 bases long, about 40 bases long, about 45 bases long, about 50 bases long, about 55 bases long, about 60 bases long, about 65 bases long, about 70 bases long, about 75 bases long, about 80 bases long, about 85 bases long, about 90 bases long, about 95 bases long, about 100 bases long, about 110 bases long, about 120 bases long, about 130, about 140 bases long, about 150 bases long, about 200 bases long, about 250 bases long, about 300 bases long, about 350 bases long, about 400 bases long, about 450 bases long, about 500 bases long, about 600 bases long, about 700 bases long, about 800 bases long, about 900 bases long, about 1000 bases long, or more than 1000 bases long.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

What is claimed is:

1. A method for detecting a genetic feature in a nucleotide sequence, the method comprising:
(a) analyzing aligned reads from the nucleotide sequence for at least one statistical feature selected from the group consisting of:
percent AT content,
percent GC content,
percent of soft clips,
percent of hard clips,
percent of reads with insert size greater than q₀ quartile,
percent of reads with insert size less than q₀ quartile,
percent of positive strand reads,
percent of negative strand reads,
percent of reads with a correct orientation,
percent of reads with a 0 x 1 BAM flag,
percent of reads with a 0 x 2 BAM flag,
percent of reads with a 0 x 4 BAM flag,
percent of reads with a 0 x 8 BAM flag,
percent of reads with a 0 x 20 BAM flag,
percent of reads with a 0 x 40 BAM flag,
percent of reads with a 0 x 80 BAM flag,
percent of reads with a 0 x 100 BAM flag,
percent of reads with a 0 x 200 BAM flag,
percent of reads with a 0 x 400 BAM flag, and
percent of reads with a 0 x 800 BAM flag; and
(b) based on the analyzing, determining a presence of the genetic feature in the nucleotide sequence.

2. The method of claim 1, wherein the aligned reads are contained in an unsorted file.

3. The method of claim 1, wherein the analyzing is performed using a programmed computer.

4. The method of claim 1, wherein the analyzing is performed using a trained algorithm.

5. The method of claim 4, wherein the trained algorithm comprises a random forest algorithm.

6. The method of claim 4, wherein the trained algorithm is trained using a moving window.

7. The method of claim 1, wherein the analyzing is performed using a moving window.

8. The method of claim 6 or 7, wherein the moving window has a length of about 50 bp.

9. The method of claim 6 or 7, wherein the moving window has a variable length.

10. The method of claim 6 or 7, wherein the analyzing does not include portions of the aligned reads located outside the moving window.

11. The method of claim 1, wherein the at least one statistical feature comprises at least two statistical features.

12. The method of claim 1, wherein the at least one statistical feature comprises at least five statistical features.

13. The method of claim 1, wherein the presence of the genetic feature is determined within a window of about 50 base pairs (bp).

14. The method of claim 1, wherein the genetic feature is a structural variant.

15. The method of claim 1, wherein the structural variant is selected from the group consisting of deletions, insertions, and inversions.

16. The method of claim 1, wherein the genetic feature is a pathogenicity marker.

17. The method of claim 1, wherein the genetic feature is a resistance marker.

18. The method of claim 1, wherein the genetic feature is a susceptibility marker.

19. The method of claim 1, wherein the genetic feature is a taxonomic marker.

20. The method of claim 1, wherein the genetic feature is from about 6 base pairs (bp) to about 50 bp in length.

21. The method of claim 1, wherein the genetic feature is from about 50 base pairs (bp) to about 500 bp in length.

22. The method of claim 1, wherein the genetic feature is greater than about 500 base pairs in length.

23. The method of claim 1, wherein the presence of the genetic feature is determined with at least 95% confidence.

24. The method of claim 1, wherein the presence of the genetic feature is determined with at least 95% accuracy.

25. The method of claim 1, wherein the presence of the genetic feature is determined with at least 95% specificity.

26. The method of claim 1, wherein the presence of the genetic feature is determined with at least 95% sensitivity.

27. The method of claim 1, wherein the determining the presence of the genetic feature comprises determining the presence of a start or an end of the genetic feature.

28. The method of claim 1, wherein the aligned reads comprise graph aligned reads.

29. The method of claim 28, wherein the analyzing is performed using a moving window, further comprising analyzing the graph aligned reads for at least one statistical feature selected from the group consisting of:
percent of paths or bubbles that fall within the window,
percent of start of the path or bubbles that fall within the window,
percent of ends of the path or bubbles that fall within the window,
percent of complete sections of the path or bubbles that fall within the window,
mean depth for each path or bubble that fall within the window,
a statistical significance of each path or bubble that falls within the window,
a portion of a total length of each path or bubble that falls within the window, and
VCF file information for each path or bubble that falls within the window.

30. The method of claim 1, wherein the aligned reads align to regions with no alternative paths.

31. The method of claim 1, wherein the aligned reads align to regions with no bubbles.

32. The method of claim 1, wherein the aligned reads align to regions with at least one alternative path or bubble.

33. A method for detecting a genetic feature in a nucleotide sequence, comprising:
(a) analyzing aligned reads from the nucleotide sequence for at least one statistical feature; and
(b) based on the analyzing, determining a presence of the genetic feature in the nucleotide sequence, wherein the genetic feature is a structural variant selected from the group consisting of an insertion, a deletion, and an inversion.

34. The method of claim 33, wherein the aligned reads are contained in an unsorted file.

35. The method of claim 33, wherein the genetic feature is from about 6 base pairs (bp) to about 50 bp in length.

36. The method of claim 33, wherein the genetic feature is from about 50 base pairs (bp) to about 500 bp in length.

37. The method of claim 33, wherein the genetic feature is greater than about 500 base pairs in length.

38. The method of claim 33, wherein the analyzing is performed using a programmed computer.

39. The method of claim 33, wherein the analyzing is performed using a trained algorithm.

40. The method of claim 39, wherein the trained algorithm comprises a random forest algorithm.

41. The method of claim 39, wherein the trained algorithm is trained using a moving window.

42. The method of claim 33, wherein the analyzing is performed using a moving window.

43. The method of claim 41 or 42, wherein the moving window has a length of about 50 bp.

44. The method of claim 41 or 42, wherein the moving window has a variable length.

45. The method of claim 41 or 42, wherein the analyzing does not include portions of the aligned reads located outside the moving window.

46. The method of claim 33, wherein the at least one statistical feature comprises at least two statistical features.

47. The method of claim 33, wherein the at least one statistical feature comprises at least five statistical features.

48. The method of claim 33, wherein the presence of the genetic feature is determined within a window of about 50 base pairs (bp).

49. The method of claim 33, wherein the at least one statistical feature is selected from the group consisting of:
percent AT content,
percent GC content,
percent of soft clips,
percent of hard clips,
percent of reads with insert size greater than q₀ quartile,
percent of reads with insert size less than q₀ quartile,
percent of positive strand reads,
percent of negative strand reads,
percent of reads with a correct orientation,
percent of reads with a 0 x 1 BAM flag,
percent of reads with a 0 x 2 BAM flag,
percent of reads with a 0 x 4 BAM flag,
percent of reads with a 0 x 8 BAM flag,
percent of reads with a 0 x 20 BAM flag,
percent of reads with a 0 x 40 BAM flag,
percent of reads with a 0 x 80 BAM flag,
percent of reads with a 0 x 100 BAM flag,
percent of reads with a 0 x 200 BAM flag,
percent of reads with a 0 x 400 BAM flag, and
percent of reads with a 0 x 800 BAM flag.

50. The method of claim 33, wherein the at least one statistical feature is selected from the group consisting of:
number of paths or bubbles that fall within a window of width w,
number of beginnings of paths or bubbles that fall within a window of width w,
number of ends of paths or bubbles that fall within a window of width w,
number of complete sections of paths or bubbles that fall within a window of width w,
mean depth of paths or bubbles that fall within a window of width w,
significance of paths or bubbles that fall within a window of width w,
portion of a total length of each path of bubble that falls within a window of width w, and
VCF file information for each path or bubble that falls within a window of width w.

51. The method of claim 33, wherein the presence of the genetic feature is determined with at least 95% confidence.

52. The method of claim 33, wherein the presence of the genetic feature is determined with at least 95% accuracy.

53. The method of claim 33, wherein the presence of the genetic feature is determined with at least 95% specificity.

54. The method of claim 33, wherein the presence of the genetic feature is determined with at least 95% sensitivity.

55. The method of claim 33, wherein the determining the presence of the genetic feature comprises determining the presence of a start or an end of the genetic feature.

56. The method of claim 33, wherein the aligned reads comprise graph aligned reads.

57. The method of claim 56, wherein the analyzing is performed using a moving window, further comprising analyzing the graph aligned reads for at least one statistical feature selected from the group consisting of:
percent of paths or bubbles that fall within the window,
percent of start of the path or bubbles that fall within the window,
percent of ends of the path or bubbles that fall within the window,
percent of complete sections of the path or bubbles that fall within the window,
mean depth for each path or bubble that fall within the window,
a statistical significance of each path or bubble that falls within the window,
a portion of a total length of each path or bubble that falls within the window, and
VCF file information for each path or bubble that falls within the window.

58. The method of claim 33, wherein the aligned reads align to regions with no alternative paths.

59. The method of claim 33, wherein the aligned reads align to regions with no bubbles.

60. The method of claim 33, wherein the aligned reads align to regions with at least one alternative path or bubble.

61. A method for detecting a genetic feature in a nucleotide sequence, comprising:
(a) analyzing aligned reads from the nucleotide sequence for at least one statistical feature, wherein the analyzing is performed using a trained algorithm that employs a moving window, and wherein the analyzing does not include portions of the aligned reads located outside the moving window; and
(b) based on the analyzing, determining a presence of the genetic feature in the nucleotide sequence.

62. The method of claim 61, wherein the aligned reads are contained in an unsorted file.

63. The method of claim 61, wherein the genetic feature is from about 6 base pairs (bp) to about 50 bp in length.

64. The method of claim 61, wherein the genetic feature is from about 50 base pairs (bp) to about 500 bp in length.

65. The method of claim 61, wherein the genetic feature is greater than about 500 base pairs in length.

66. The method of claim 61, wherein the analyzing is performed using a programmed computer.

67. The method of claim 61, wherein the trained algorithm comprises a random forest algorithm.

68. The method of claim 61, wherein the genetic feature is a structural variant.

69. The method of claim 68, wherein the structural variant is selected from the group consisting of deletions, insertions, and inversions.

70. The method of claim 61, wherein the genetic feature is a pathogenicity marker.

71. The method of claim 61, wherein the genetic feature is a resistance marker.

72. The method of claim 61, wherein the genetic feature is a susceptibility marker.

73. The method of claim 61, wherein the genetic feature is a taxonomic marker.

74. The method of claim 61, wherein the moving window has a length of about 50 bp.

75. The method of claim 61, wherein the moving window has a variable length.

76. The method of claim 61, wherein the at least one statistical feature comprises at least two statistical features.

77. The method of claim 61, wherein the at least one statistical feature comprises at least five statistical features.

78. The method of claim 61, wherein the presence of the genetic feature is determined within a window of about 50 base pairs (bp).

79. The method of claim 61, wherein the at least one statistical feature is selected from the group consisting of:
percent AT content,
percent GC content,
percent of soft clips,
percent of hard clips,
percent of reads with insert size greater than q₀ quartile,
percent of reads with insert size less than q₀ quartile,
percent of positive strand reads,
percent of negative strand reads,
percent of reads with a correct orientation,
percent of reads with a 0 x 1 BAM flag,
percent of reads with a 0 x 2 BAM flag,
percent of reads with a 0 x 4 BAM flag,
percent of reads with a 0 x 8 BAM flag,
percent of reads with a 0 x 20 BAM flag,
percent of reads with a 0 x 40 BAM flag,
percent of reads with a 0 x 80 BAM flag,
percent of reads with a 0 x 100 BAM flag,
percent of reads with a 0 x 200 BAM flag,
percent of reads with a 0 x 400 BAM flag, and
percent of reads with a 0 x 800 BAM flag.

80. The method of claim 61, wherein the at least one statistical feature is selected from the group consisting of:
number of paths or bubbles that fall within a window of width w,
number of beginnings of paths or bubbles that fall within a window of width w,
number of ends of paths or bubbles that fall within a window of width w,
number of complete sections of paths or bubbles that fall within a window of width w,
mean depth of paths or bubbles that fall within a window of width w,
significance of paths or bubbles that fall within a window of width w,
portion of a total length of each path of bubble that falls within a window of width w, and
VCF file information for each path or bubble that falls within a window of width w.

81. The method of claim 61, wherein the presence of the genetic feature is determined with at least 95% confidence.

82. The method of claim 61, wherein the presence of the genetic feature is determined with at least 95% accuracy.

83. The method of claim 61, wherein the presence of the genetic feature is determined with at least 95% specificity.

84. The method of claim 61, wherein the presence of the genetic feature is determined with at least 95% sensitivity.

85. The method of claim 61, wherein the determining the presence of the genetic feature comprises determining the presence of a start or an end of the genetic feature.

86. The method of claim 61, wherein the aligned reads comprise graph aligned reads.

87. The method of claim 86, wherein the analyzing is performed using a moving window, further comprising analyzing the graph aligned reads for at least one statistical feature selected from the group consisting of:
percent of paths or bubbles that fall within the window,
percent of start of the path or bubbles that fall within the window,
percent of ends of the path or bubbles that fall within the window,
percent of complete sections of the path or bubbles that fall within the window,
mean depth for each path or bubble that fall within the window,
a statistical significance of each path or bubble that falls within the window,
a portion of a total length of each path or bubble that falls within the window, and
VCF file information for each path or bubble that falls within the window.

88. The method of claim 61, wherein the aligned reads align to regions with no alternative paths.

89. The method of claim 61, wherein the aligned reads align to regions with no bubbles.

90. The method of claim 61, wherein the aligned reads align to regions with at least one alternative path or bubble.

91. A method for detecting a genetic feature in a nucleotide sequence, comprising:
(a) analyzing graph aligned reads from the nucleotide sequence for at least one statistical feature; and
(b) based on the analyzing, determining a presence of the genetic feature in the nucleotide sequence.

92. The method of claim 91, wherein the aligned reads are contained in an unsorted file.

93. The method of claim 91, wherein the genetic feature is from about 6 base pairs (bp) to about 50 bp in length.

94. The method of claim 91, wherein the genetic feature is from about 50 base pairs (bp) to about 500 bp in length.

95. The method of claim 91, wherein the genetic feature is greater than about 500 base pairs in length.

96. The method of claim 91, wherein the analyzing is performed using a programmed computer.

97. The method of claim 91, wherein the analyzing is performed using a trained algorithm.

98. The method of claim 97, wherein the trained algorithm comprises a random forest algorithm.

99. The method of claim 97, wherein the trained algorithm is trained using a moving window.

100. The method of claim 91, wherein the analyzing is performed using a moving window.

101. The method of claim 99 or 100, wherein the moving window has a length of about 50 bp.

102. The method of claim 99 or 100, wherein the moving window has a variable length.

103. The method of claim 99 or 100, wherein the analyzing does not include portions of the aligned reads located outside the moving window.

104. The method of claim 91, wherein the at least one statistical feature comprises at least two statistical features.

105. The method of claim 91, wherein the at least one statistical feature comprises at least five statistical features.

106. The method of claim 91, wherein the presence of the genetic feature is determined within a window of about 50 base pairs (bp).

107. The method of claim 91, wherein the at least one statistical feature is selected from the group consisting of:
percent AT content,
percent GC content,
percent of soft clips,
percent of hard clips,
percent of reads with insert size greater than q₀ quartile,
percent of reads with insert size less than q₀ quartile,
percent of positive strand reads,
percent of negative strand reads,
percent of reads with a correct orientation,
percent of reads with a 0 x 1 BAM flag,
percent of reads with a 0 x 2 BAM flag,
percent of reads with a 0 x 4 BAM flag,
percent of reads with a 0 x 8 BAM flag,
percent of reads with a 0 x 20 BAM flag,
percent of reads with a 0 x 40 BAM flag,
percent of reads with a 0 x 80 BAM flag,
percent of reads with a 0 x 100 BAM flag,
percent of reads with a 0 x 200 BAM flag,
percent of reads with a 0 x 400 BAM flag, and
percent of reads with a 0 x 800 BAM flag.

108. The method of claim 91, wherein the at least one statistical feature is selected from the group consisting of:
number of paths or bubbles that fall within a window of width w,
number of beginnings of paths or bubbles that fall within a window of width w,
number of ends of paths or bubbles that fall within a window of width w,
number of complete sections of paths or bubbles that fall within a window of width w,
mean depth of paths or bubbles that fall within a window of width w,
significance of paths or bubbles that fall within a window of width w,
portion of a total length of each path of bubble that falls within a window of width w, and
VCF file information for each path or bubble that falls within a window of width w.

109. The method of claim 91, wherein the presence of the genetic feature is determined with at least 95% confidence.

110. The method of claim 91, wherein the presence of the genetic feature is determined with at least 95% accuracy.

111. The method of claim 91, wherein the presence of the genetic feature is determined with at least 95% specificity.

112. The method of claim 91, wherein the presence of the genetic feature is determined with at least 95% sensitivity.

113. The method of claim 91, wherein the determining the presence of the genetic feature comprises determining the presence of a start or an end of the genetic feature.

114. The method of claim 91, wherein the genetic feature is a structural variant.

115. The method of claim 114, wherein the structural variant is selected from the group consisting of an insertion, a deletion, and an inversion.

116. The method of claim 91, wherein the genetic feature is a pathogenicity marker.

117. The method of claim 91, wherein the genetic feature is a resistance marker.

118. The method of claim 91, wherein the genetic feature is a susceptibility marker.

119. The method of claim 91, wherein the genetic feature is a taxonomic marker.

120. The method of claim 91, wherein the aligned reads align to regions with no alternative paths.

121. The method of claim 91, wherein the graph aligned reads align to regions with no bubbles.

122. The method of claim 91, wherein the graph aligned reads align to regions with at least one alternative path or bubble.

123. A method for detecting a genetic feature in a nucleotide sequence, comprising:
(a) analyzing, using a moving window, aligned reads from the nucleotide sequence for at least one statistical feature selected from the group consisting of:
number of paths or bubbles that fall within the window,
number of beginnings of paths or bubbles that fall within the window,
number of ends of paths or bubbles that fall within the window,
number of complete sections of paths or bubbles that fall within the window,
mean depth of paths or bubbles that fall within the window,
significance of paths or bubbles that fall within the window,
portion of a total length of each path of bubble that falls within the window, and
VCF file information for each path or bubble that falls within the window; and
(b) based on the analyzing, determining a presence of the genetic feature in the nucleotide sequence.

124. The method of claim 123, wherein the aligned reads are contained in an unsorted file.

125. The method of claim 123, wherein the analyzing is performed using a programmed computer.

126. The method of claim 123, wherein the analyzing is performed using a trained algorithm.

127. The method of claim 126, wherein the trained algorithm comprises a random forest algorithm.

128. The method of claim 126, wherein the trained algorithm is trained using a moving window.

129. The method of claim 123, wherein the analyzing is performed using a moving window.

130. The method of claim 128 or 129, wherein the moving window has a length of about 50 bp.

131. The method of claim 128 or 129, wherein the moving window has a variable length.

132. The method of claim 128 or 129, wherein the analyzing does not include portions of the aligned reads located outside the moving window.

133. The method of claim 123, wherein the at least one statistical feature comprises at least two statistical features.

134. The method of claim 123, wherein the at least one statistical feature comprises at least five statistical features.

135. The method of claim 123, wherein the presence of the genetic feature is determined within a window of about 50 base pairs (bp).

136. The method of claim 123, wherein the genetic feature is a structural variant.

137. The method of claim 137, wherein the structural variant is selected from the group consisting of deletions, insertions, and inversions.

138. The method of claim 123, wherein the genetic feature is a pathogenicity marker.

139. The method of claim 123, wherein the genetic feature is a resistance marker.

140. The method of claim 123, wherein the genetic feature is a susceptibility marker.

141. The method of claim 123, wherein the genetic feature is a taxonomic marker.

142. The method of claim 123, wherein the genetic feature is from about 6 base pairs (bp) to about 50 bp in length.

143. The method of claim 123, wherein the genetic feature is from about 50 base pairs (bp) to about 500 bp in length.

144. The method of claim 123, wherein the genetic feature is greater than about 500 base pairs in length.

145. The method of claim 123, wherein the presence of the genetic feature is determined with at least 95% confidence.

146. The method of claim 123, wherein the presence of the genetic feature is determined with at least 95% accuracy.

147. The method of claim 123, wherein the presence of the genetic feature is determined with at least 95% specificity.

148. The method of claim 123, wherein the presence of the genetic feature is determined with at least 95% sensitivity.

149. The method of claim 123, wherein the determining the presence of the genetic feature comprises determining the presence of a start or an end of the genetic feature.

150. The method of claim 123, wherein the aligned reads comprise graph aligned reads.

151. The method of claim 150, wherein the analyzing is performed using a moving window, further comprising analyzing the graph aligned reads for at least one statistical feature selected from the group consisting of:
percent of paths or bubbles that fall within the window,
percent of start of the path or bubbles that fall within the window,
percent of ends of the path or bubbles that fall within the window,
percent of complete sections of the path or bubbles that fall within the window,
mean depth for each path or bubble that fall within the window,
a statistical significance of each path or bubble that falls within the window,
a portion of a total length of each path or bubble that falls within the window, and
VCF file information for each path or bubble that falls within the window.

152. The method of claim 123, wherein the aligned reads align to regions with no alternative paths.

153. The method of claim 123, wherein the aligned reads align to regions with no bubbles.

154. The method of claim 123, wherein the aligned reads align to regions with at least one alternative path or bubble.

155. The method of claim 123, further comprising analyzing aligned reads from the nucleotide sequence for at least one statistical feature selected from the group consisting of:
percent AT content,
percent GC content,
percent of soft clips,
percent of hard clips,
percent of reads with insert size greater than q₀ quartile,
percent of reads with insert size less than q₀ quartile,
percent of positive strand reads,
percent of negative strand reads,
percent of reads with a correct orientation,
percent of reads with a 0 x 1 BAM flag,
percent of reads with a 0 x 2 BAM flag,
percent of reads with a 0 x 4 BAM flag,
percent of reads with a 0 x 8 BAM flag,
percent of reads with a 0 x 20 BAM flag,
percent of reads with a 0 x 40 BAM flag,
percent of reads with a 0 x 80 BAM flag,
percent of reads with a 0 x 100 BAM flag,
percent of reads with a 0 x 200 BAM flag,
percent of reads with a 0 x 400 BAM flag, and
percent of reads with a 0 x 800 BAM flag.

156. A method for detecting a genetic feature in a nucleotide sequence, comprising:
(a) analyzing aligned reads from the nucleotide sequence for at least one statistical feature selected from the group consisting of input information depth, coverage, orientation of the aligned reads, and insert size between paired-end reads; and
(b) based on the analyzing, determining a presence of the genetic feature.

157. The method of claim 156, wherein the aligned reads are contained in an unsorted file.

158. The method of claim 156, wherein the genetic feature is a clade marker.

159. The method of claim 157, wherein the clade marker is a pathogen clade marker.

160. The method of claim 157, wherein the clade marker is a bacteria clade marker.

161. The method of claim 157, wherein the clade marker is a virus clade marker.

162. The method of claim 157, wherein the clade marker is a fungus clade marker.

163. The method of claim 157, wherein the clade marker is a protozoa clade marker.

164. The method of claim 156, wherein the genetic feature is a structural variant.

165. The method of claim 164, wherein the structural variant is an insertion.

166. The method of claim 164, wherein the structural variant is a deletion.

167. The method of claim 164, wherein the structural variant is a copy number variation.

168. The method of claim 164, wherein the structural variant is an inversion.

169. The method of claim 156, further comprising, based on the analyzing, determining a location of the genetic feature.

170. The method of claim 169, further comprising determining a confidence value of the location of the genetic feature.

171. The method of claim 169, wherein the genetic feature is a structural variant.

172. The method of claim 169, wherein the genetic feature is a flanking region.

173. The method of claim 156, wherein the analyzing is performed using a moving window.

174. The method of claim 173, wherein the moving window has a variable length.

175. The method of claim 173, wherein the analyzing does not include portions of the aligned reads located outside of the moving window.

176. The method of claim 156, wherein the aligned reads comprise graph aligned reads.

177. The method of claim 156, wherein the aligned reads align to regions with no alternative paths.

178. The method of claim 156, wherein the aligned reads align to regions with no bubbles.

179. The method of claim 156, wherein the aligned reads align to regions with at least one alternative path or bubble.

180. A method for locating a genetic feature in a nucleotide sequence, comprising:
(a) analyzing prior information, the prior information comprising (i) genetic feature population information or (ii) genetic feature reference information;
(b) analyzing genetic feature presence information;
(c) based on the analyzing in (a) and the analyzing in (b), determining a location of the genetic feature.

181. The method of claim 180, wherein the aligned reads are contained in an unsorted file.

182. The method of claim 180, wherein the genetic feature presence information is determined by analyzing aligned reads from the nucleotide sequence for at least one statistical feature and, based on the analyzing, determining the presence of the genetic feature.

183. The method of claim 180, further comprising determining a confidence value of the location of the genetic feature.

184. The method of claim 180, wherein the genetic feature is a clade marker.

185. The method of claim 184, wherein the clade marker is a pathogen clade marker.

186. The method of claim 184, wherein the clade marker is a bacteria clade marker.

187. The method of claim 184, wherein the clade marker is a virus clade marker.

188. The method of claim 184, wherein the clade marker is a fungus clade marker.

189. The method of claim 184, wherein the clade marker is a protozoa clade marker.

190. The method of claim 180, wherein the genetic feature is a structural variant.

191. The method of claim 190, wherein the structural variant is an insertion.

192. The method of claim 190, wherein the structural variant is a deletion.

193. The method of claim 190, wherein the structural variant is a copy number variation.

194. The method of claim 190, wherein the structural variant is an inversion.

195. The method of claim 180, further comprising determining a location of each of a plurality of genetic features.

196. The method of claim 195, further comprising a confidence value for each location of the plurality of genetic features.

197. The method of claim 195, further comprising determining a genomic structure of the plurality of genetic features.
